(19) United States
(12) Patent Application Publication
(10) Pub. No.: US 2005/0032074 A1
(43) Pub. Date: Feb. 10, 2005

(54) Custom design method for resequencing arrays

(75) Inventors: Nila A. Shah, Cupertino, CA (US); Janet A. Warrington, Los Altos, CA (US); Xue Mei Zhou, San Jose, CA (US); Michael P. Mittmann, Palo Alto, CA (US); Rui Mei, Santa Clara, CA (US)

Correspondence Address: AFFYMETRIX, INC;ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA (US)

(21) Appl. No.: 10/658,879

(22) Filed: Sep. 9, 2003

Related U.S. Application Data
(60) Provisional application No. 60/409,396, filed on Sep. 9, 2002.
(60) Provisional application No. 60/409,800, filed on Sep. 11, 2002.

(51) Int. Cl. G06F 17/60; C12Q 1/68; G06F 19/00; G01N 33/48; G01N 33/50

(52) U.S. Cl. 435/6; 702/20; 705/3

(57) ABSTRACT

A method is described that receives user-selected sequence and provides a custom array designed for resequencing the user-selected sequence. The system determines probes corresponding to the sequence and to possible variants of the sequence and generates a custom probe array design. The system then displays the custom probe array design to the user via a graphical user interface and receives a user acceptance of the design. The system provides the user with the accepted custom probe array. The system may also enable a number of users to share space on a custom probe array. Another optional feature is to enable a number of users to share in ordering portions of a lot of catalog probe arrays to take advantage of economies of scale from lot-size purchases.

### RELATED APPLICATIONS

This application claims priority to Provisional Application No. 60/409,396 filed Sep. 9, 2002 and Provisional Application No. 60/409,800, filed Sep. 11, 2002, the disclosures of which are incorporated herein by reference in their entireties.

### FIELD OF THE INVENTION

The present invention relates to the field of analysis of genomic information using microarrays. In particular, methods for designing microarrays for resequencing genomic regions and methods for a provider of microarrays or microarray designs to interact with a customer/user desiring a microarray or microarray design with specified features are disclosed.

### BACKGROUND

The past years have seen a dynamic change in the ability of science to comprehend vast amounts of data. Pioneering technologies such as nucleic acid arrays allow scientists to delve into the world of genetics in far greater detail than ever before. Exploration of genomic DNA has long been a dream of the scientific community. Held within the complex structures of genomic DNA lies the potential to identify, diagnose, or treat diseases like cancer, Alzheimer disease or alcoholism. Exploitation of genomic information from plants and animals may also provide answers to the world's food distribution problems.

Genomic variation between individuals is believed to account for more than 90% of all differences between individuals. This variation is typically found in the form of polymorphisms with single nucleotide polymorphisms (SNPs) accounting for the majority of genetic variation. Understanding the relationship between genetic variation and biological function on a genomic scale is expected to provide insight into the biology of humans and other species, including those that cause disease in humans. Identification of large numbers of SNPs will be integral to furthering our understanding.

### SUMMARY

Methods for using a computer to design a resequencing array to resequence a user selected sequence are disclosed. In a preferred embodiment a design request comprising a user selected sequence, which may be in the form of a sequence file, is received by the provider and the provider produces an array design for resequencing that user selected sequence. The design is output to said user, for example, over the internet and the user checks the design and can either accept, reject or suggest modifications to the design. If the user accepts the design then the provider generates a file that may be used to instruct a nucleic acid synthesizer, which may or may not use masks, to synthesize the array on an appropriate support. The design may also include appropriate probes which may be selected by the user or by the provider. The synthesized array is provided to the customer. The customer then may do long range PCR to amplify the selected sequence from a sample from an individual and can then hybridize the amplicons to the array, detect the hybridization pattern and determining the sequence of the individual.

In another embodiment a method for a provider of nucleic acid arrays receivs a sequence computer file from a user wherein the sequence computer file comprises the sequence of the user selected nucleic acid and the provider prepares a design for a resequencing array for the selected sequence. The provider outputs a design into a design computer file and provides the design computer file to the user and receives user approval for the design before outputting an instruction computer file wherein the instruction computer file provides instructions to a nucleic acid synthesizer for synthesis of an array comprising said design. The provider then synthesizes the array; and provides the user with the array. The provider may further analyze the sequence file from the user to remove repetitive sequences, to identify and optionally remove homologous sequence that may cross hybridize to the same probes, and to remove sequence ambiguities. Sequence ambiguities occur when a genomic region is sequenced on more than one occasion or from more than one source and differences occur in the resulting sequence. These differences may be the result of sequence error or from polymorphism. Comparison of the same sequence from multiple sources may be used to identify sequence errors and those can be removed from the design. Ambiguity may be removed by the provider or by the customer with or without the assistance of the provider. The provider may provide software applications to assist the user in preparing sequence for design of a resequencing array. The applications may be provided on the internet.

In one embodiment the provider also provides sequences of primers to be used for amplification of sequence for resequencing analysis. The provider may also provide a graphical user interface whereby the customer may order primers from a third party over the internet.

In one embodiment the user first identifies a sequence for resequencing using an association study or linkage study using a genotyping array, such as the Mapping 10 K Array, available from Affymetrix, Inc. The Mapping 10 K Array may be used to genotype more than 10,000 human SNPs. Genomic regions that are linked to a selected phenotype, such as a disease, may be identified by genotyping a plurality of affected and unaffected ndividuals at a plurality of SNPs that are spaced throughout the genome. The genomic region or regions that are identified in this manner are then candidates for a finer level of mapping, for example, the region or regions may be resequenced to identify the genotype of all SNPs in the region. Resequencing may also be used to identify novel SNPs, including SNPs that occur at very low frequencies. In this manner customers may identify SNPs that are tightly linked to a phenotype and may be diagnostic of that phenotype or may contribute to the phenotype.

In one embodiment the provider pre-designs arrays for regions of the genome that the provider anticipates may be of interest to one or more customer. In a preferred embodiment the provider has arrays designed for all or most regions of a genome of interest, including the human genome. The arrays are preferably designed by a computer. The designs available would be available to the customer in a list that may be available on the internet. A customer may then order an array from the list. The provider would then synthesize the arrays ordered by the customer by outputting instructions to a nucleic acid synthesizer. The provider would then provide the arrays to the customer. The provider may also provide recommended primer sequences that may be used with the selected arrays to amplify the sequences interrogated by the array(s) ordered by the user. In one embodiment the customer may order, for example, one or more arrays that interrogate a region of a chromosome, an entire chromosome.

### BRIEF DESCRIPTION OF THE DRAWINGS

FIG. 1 shows a flow chart of the steps in the process of ordering, designing and delivering a custom resequencing array.

### DETAILED DESCRIPTION

(A) General

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach”1984, IRL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W. H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285 (International Publication Number WO 01/58593), which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, 5,959,098, 6,271,957 and 6,480,324, each of which is incorporated herein by reference in its entirety for all purposes. Nucleic acid arrays are described in many of the above patents, but the same techniques are applied to polypeptide arrays. Methods for synthesizing a maskless array using programmable micro-mirror are described in U.S. Pat. Nos. 6,271,957 and 6,480,324

Nucleic acid arrays that are useful in the present invention include those that are commercially available from Affymetrix (Santa Clara, Calif.) under the brand name GeneChip®. Example arrays are shown on the website at affymetrix.com.

The present invention also contemplates many uses for polymers attached to solid substrates. These uses include gene expression monitoring, profiling, library screening, genotyping and diagnostics. Gene expression monitoring, and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. Nos. 60/319,253, 10/013,598 (U.S. Patent Application Publication 20030036069), and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Ed. Innis, et al., Academic Press, San Diego, Calif. 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No. 6,300,070 and U.S. Ser. No. 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No 6,410,276), consensus sequence primed polymerase chain reaction (CP-PCR) (U.S. Pat. No. 4,437,975), arbitrarily primed polymerase chain reaction (AP-PCR) (U.S. Pat. No. 5, 413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. No. 6,361,947, 6,391,592 and U.S. Ser. Nos. 09/916,135, 09/920,491 (U.S. patent Application Publication 20030096235), 09/910,292 (U.S. patent Application Publication 20030082543), and 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y, 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference.

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Ser. No. 60/364,731 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Ser. No. 60/364,731 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001). See U.S. Pat. No. 6,420,108.

Computer software products may be written in any of various suitable programming languages, such as C, C++, Fortran and Java (Sun Microsystems). The computer software product may be an independent application with data input and data display modules. Alternatively, the computer software products may be classes that may be instantiated as distributed objects. The computer software products may also be component software such as Java Beans (Sun Microsystems), Enterprise Java Beans (EJB), Microsoft.RTM. COM/DCOM, etc.

Systems, methods, and computer products are now described with reference to an illustrative embodiment referred to as a genomic portal. A portal may be an Internet environment. In a typical implementation, the portal may be used to provide a user with information related to results from experiments with probe arrays. The experiments often involve the use of scanning equipment to detect hybridization of probe-target pairs, and the analysis of detected hybridization by various software applications.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. Ser. Nos. 10/063,559 (United States Publication No. 20020183936), 60/349,546, 60/376,003, 60/394,574 and 60/403,381.

(B) Definitions

An array is an intentionally created collection of molecules which can be prepared either synthetically or biosynthetically. The molecules in the array can be identical or different from each other. The array can assume a variety of formats, e.g., libraries of soluble molecules; libraries of compounds tethered to resin beads, silica chips, or other solid supports.

A genotyping array comprises probes that are specific for one allele of a polymorphism. Genotyping arrays are described, for example, in U.S. patent application Nos. 10/264,945 and 10/442,021 and U.S. Provisional patent applications Nos. 60/470,475 filed 5/14/2003, 60/483,050 filed 6/27/2003 and 60/417,190 filed 10/8/2002, each of which is incorporated herein by reference in its entirety.

Combinatorial chemistry may be used for the parallel synthesis of discreet compounds, for example, oligonucleotides of different sequence on a solid support. See, for example, U.S. Pat. Nos. 5,412,087, 5,424,186, 5,445,934 and 6,040,193 which are each incorporated herein by reference. Many different compounds may be synthesized. The compounds may be oligonucleotides which may be synthesized on a solid support so that each discreet compound or oligonucleotide is localized to a specific region, or feature, of the array which may be predefined. In some embodiments there may be overlap between regions. In some embodiments a plurality of different oligonucleotides are generated, each sharing a core set of bases but differing at some positions. For example, each feature of the array may be of the sequence 5′-GAATNNCNG-3′ and within each discreet feature the N's will be the same, for example one feature may be 5′-GAATcgCtG-3′ while another feature may be 5′-GAATttCgG-3′. The core bases are GAAT-C-G. The synthesis of many different probes may be accomplished in this manner with increase efficiency and decreased cost because the majority of probes of the array have the same core set of bases and addition of those bases may be done en masse without the use of feature specific photolithography, i.e. all features may be activated simultaneously for those positions. In some embodiments there are control oligonucleotides on the array that may or may not share the common core bases.

Nucleic acid library or array is an intentionally created collection of nucleic acids which can be prepared either synthetically or biosynthetically and screened for biological activity in a variety of different formats (e.g., libraries of soluble molecules; and libraries of oligos tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

Biopolymer or biological polymer is intended to mean repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above. Biopolymer synthesis is intended to encompass the synthetic production, both organic and inorganic, of a biopolymer.

Related to a bioploymer is a biomonomer which is intended to mean a single unit of biopolymer, or a single unit which is not part of a biopolymer. Thus, for example, a nucleotide is a biomonomer within an oligonucleotide biopolymer, and an amino acid is a biomonomer within a protein or peptide biopolymer; avidin, biotin, antibodies, antibody fragments, etc., for example, are also biomonomers. Initiation Biomonomer or initiator biomonomer is meant to indicate the first biomonomer which is covalently attached via reactive nucleophiles to the surface of the polymer, or the first biomonomer which is attached to a linker or spacer arm attached to the polymer, the linker or spacer arm being attached to the polymer via reactive nucleophiles.

Complementary or substantially complementary refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementary exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

A combinatorial synthesis strategy is an ordered strategy for parallel synthesis of diverse polymer sequences by sequential addition of reagents which may be represented by a reactant matrix and a switch matrix, the product of which is a product matrix. A reactant matrix is a 1 column by m row matrix of the building blocks to be added. The switch matrix is all or a subset of the binary numbers, preferably ordered, between 1 and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids.

Effective amount refers to an amount sufficient to induce a desired result.

Genome is all the genetic material in the chromosomes of an organism. DNA derived from the genetic material in the chromosomes of a particular organism is genomic DNA. A genomic library is a collection of clones made from a set of randomly generated overlapping DNA fragments representing the entire genome of an organism.

The term “hybridization” refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.”

Hybridizations are usually performed under stringent conditions, for example, at a salt concentration of no more than 1 M and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see, for example, Sambrook, Fritsche and Maniatis. “Molecular Cloning A laboratory Manual” 2^{nd} Ed. Cold Spring Harbor Press (1989) which is hereby incorporated by reference in its entirety for all purposes.

Hybridization probes are oligonucleotides capable of binding in a base-specific manner to a complementary strand of nucleic acid. Such probes include peptide nucleic acids, as described in Nielsen et al., Science 254, 1497-1500 (1991), and other nucleic acid analogs and nucleic acid mimetics. See, for example, U.S. Pat. No. 6,156,501.

Isolated nucleic acid is an object species invention that is the predominant species present (i.e., on a molar basis it is more abundant than any other individual species in the composition). Preferably, an isolated nucleic acid comprises at least about 50, 80 or 90% (on a molar basis) of all macromolecular species present. Most preferably, the object species is purified to essential homogeneity (contaminant species cannot be detected in the composition by conventional detection methods).

Ligand: A ligand is a molecule that is recognized by a particular receptor. The agent bound by or reacting with a receptor is called a “ligand,” a term which is definitionally meaningful only in terms of its counterpart receptor. The term “ligand” does not imply any particular molecular size or other structural or compositional feature other than that the substance in question is capable of binding or otherwise interacting with the receptor. Also, a ligand may serve either as the natural ligand to which the receptor binds, or as a functional analogue that may act as an agonist or antagonist. Examples of ligands that can be investigated by this invention include, but are not restricted to, agonists and antagonists for cell membrane receptors, toxins and venoms, viral epitopes, hormones (e.g., opiates, steroids, etc.), hormone receptors, peptides, enzymes, enzyme substrates, substrate analogs, transition state analogs, cofactors, drugs, proteins, and antibodies.

Linkage disequilibrium or allelic association means the preferential association of a particular allele or genetic marker with a specific allele, or genetic marker at a nearby chromosomal location more frequently than expected by chance for any particular allele frequency in the population. For example, if locus X has alleles a and b, which occur equally frequently, and linked locus Y has alleles c and d, which occur equally frequently, one would expect the combination ac to occur with a frequency of 0.25. If ac occurs more frequently, then alleles a and c are in linkage disequilibrium. Linkage disequilibrium may result from natural selection of certain combination of alleles or because an allele has been introduced into a population too recently to have reached equilibrium with linked alleles.

Determination of the genotype of variations in individuals with a selected phenotype, for example, a disease, and in unaffected controls may be used to test for association of specific alleles with a phenotype, for example, susceptibility to disease. Such studies are called association studies. These studies may be family-based, wherein members of one or more families are genotyped in order to minimize genetic diversity, or they may be performed on unrelated individuals

Mixed population or complex population: refers to any sample containing both desired and undesired nucleic acids. As a non-limiting example, a complex population of nucleic acids may be total genomic DNA, total genomic RNA or a combination thereof. Moreover, a complex population of nucleic acids may have been enriched for a given population but include other undesirable populations. For example, a complex population of nucleic acids may be a sample which has been enriched for desired messenger RNA (mRNA) sequences but still includes some undesired ribosomal RNA sequences (rRNA).

Monomer: refers to any member of the set of molecules that can be joined together to form an oligomer or polymer. The set of monomers useful in the present invention includes, but is not restricted to, for the example of (poly)peptide synthesis, the set of L-amino acids, D-amino acids, or synthetic amino acids. As used herein, “monomer” refers to any member of a basis set for synthesis of an oligomer. For example, dimers of L-amino acids form a basis set of 400 “monomers” for synthesis of polypeptides. Different basis sets of monomers may be used at successive steps in the synthesis of a polymer. The term “monomer” also refers to a chemical subunit that can be combined with a different chemical subunit to form a compound larger than either subunit alone.

mRNA or mRNA transcripts: as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

Nucleic acid library or array is an intentionally created collection of nucleic acids which can be prepared either synthetically or biosynthetically and screened for biological activity in a variety of different formats (e.g., libraries of soluble molecules; and libraries of oligos tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein.

These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

Nucleic acids according to the present invention may include any polymer or oligomer of pyrimidine and purine bases, preferably cytosine, thymine, and uracil, and adenine and guanine, respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) which may be isolated from natural sources, recombinantly produced or artificially synthesized and mimetics thereof. A further example of a polynucleotide of the present invention may be peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

Probe: A probe is a surface-immobilized molecule that can be recognized by a particular target. Examples of probes that can be investigated by this invention include, but are not restricted to, agonists and antagonists for cell membrane receptors, toxins and venoms, viral epitopes, hormones (e.g., opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

Primer is a single-stranded oligonucleotide capable of acting as a point of initiation for template-directed DNA synthesis under suitable conditions e.g., buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 30 nucleotides. Short primer molecules generally require cooler temperatures to form sufficiently stable hybrid complexes with the template. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

The term “chromosome” refers to the heredity-bearing gene carrier of a living cell which is derived from chromatin and which comprises DNA and protein components (especially histones). The conventional internationally recognized individual human genome chromosome numbering system is employed herein. The size of an individual chromosome can vary from one type to another with a given multi-chromosomal genome and from one genome to another. In the case of the human genome, the entire DNA mass of a given chromosome is usually greater than about 100,000,000 bp. For example, the size of the entire human genome is about 3×10⁹ bp. The largest chromosome, chromosome no. 1, contains about 2.4×10⁸ bp while the smallest chromosome, chromosome no. 22, contains about 5.3×10⁷ bp.

A chromosomal region is a portion of a chromosome. The actual physical size or extent of any individual chromosomal region can vary greatly. The term region is not necessarily definitive of a particular one or more genes because a region need not take into specific account the particular coding segments (exons) of an individual gene.

An allele refers to one specific form of a genetic sequence (such as a gene) within a cell, an individual or within a population, the specific form differing from other forms of the same gene in the sequence of at least one, and frequently more than one, variant sites within the sequence of the gene. The sequences at these variant sites that differ between different alleles are termed “variances”, “polymorphisms”, or “mutations”. At each autosomal specific chromosomal location or “locus” an individual possesses two alleles, one inherited from one parent and one from the other parent, for example one from the mother and one from the father. An individual is “heterozygous” at a locus if it has two different alleles at that locus. An individual is “homozygous” at a locus if it has two identical alleles at that locus.

Polymorphism refers to the occurrence of two or more genetically determined alternative sequences or alleles in a population. A polymorphic marker or site is the locus at which divergence occurs. Preferred markers have at least two alleles. Each SNP allele occurs at a given frequency which may vary from population to population. Some SNPs have alleles that occurr at a frequency of greater than 1% in a selected population, while some alleles occur at frequencies greater than 10% or 20% in a selected population. Some SNP alleles may also occur in less than 1% of a given population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. Single nucleotide polymorphisms (SNPs) are included in polymorphisms. Sequence variation may also be present in a very small percentage of a population, including in a single individual.

Receptor: A molecule that has an affinity for a given ligand. Receptors may be naturally-occurring or manmade molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term receptors is used herein, no difference in meaning is intended. A “Ligand Receptor Pair” is formed when two macromolecules have combined through molecular recognition to form a complex. Other examples of receptors which can be investigated by this invention include but are not restricted to those molecules shown in U.S. Pat. No. 5,143,854, which is hereby incorporated by reference in its entirety.

“Solid support”, “support”, and “substrate” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates.

A tag or tag sequence is a selected nucleic acid with a specified nucleic acid sequence. A tag probe has a region that is complementary to a selected tag. A set of tags or a collection of tags is a collection of specified nucleic acids that may be of similar length and similar hybridization properties, for example similar Tₘ. The tags in a collection of tags bind to tag probes with minimal cross hybridization so that a single species of tag in the tag set accounts for the majority of tags which bind to a given tag probe species under hybridization conditions. For additional description of tags and tag probes and methods of selecting tags and tag probes see U.S. Ser. No. 08/626,285 and EP/0799897, each of which is incorporated herein by reference in their entirety.

Target: A molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term “targets” is used herein, no difference in meaning is intended. A “Probe Target Pair” is formed when two macromolecules have combined through molecular recognition to form a complex.

As used herein, the term “graphical user interface” is intended to be broadly interpreted so as to include various ways of communicating information to, and obtaining information from, a user. For example, information may be sent to a user in an email as an alternative to, or in addition to, presenting the information on a computer screen employing graphical elements (such as shown illustratively in FIGS. 13A and 13B). As is known by those of ordinary skill in the relevant art, the email may include graphics, or be designed to invoke graphics, similar to those that may be displayed in an interactive graphical user interface.

A computer may be a computing device specially designed and configured to support and execute some or all of the functions of probe array applications. Computer also may be any of a variety of types of general-purpose computers such as a personal computer, network server, workstation, or other computer platform now or later developed. Computer typically includes known components such as a processor, an operating system, a graphical user interface (GUI) controller, a system memory, memory storage devices, and input-output controllers. It will be understood by those skilled in the relevant art that there are many possible configurations of the components of computer and that some may include components such as cache memory, a data backup unit, and many other devices.

A processor may be a commercially available processor such as a Pentium.RTM. processor made by Intel Corporation, a SPARC.RTM. processor made by Sun Microsystems, or it may be one of other processors that are or will become available. The processor executes an operating system, which may be, for exanple, a Windows.RTM.-type operating system (such as Windows NT.RTM.4.0 with SP6a) from the Microsoft Corporation; a Unix.RTM. or Linux-type operating system available from many vendors; another or a future operating system; or some combination thereof.

An operating system interfaces with firmware and hardware in a well-known manner, and facilitates a processor in coordinating and executing the functions of various computer programs that may be written in a variety of programming languages. An operating system, typically in cooperation with a processor, coordinates and executes functions of the other components of a computer. An operating system also provides scheduling, input-output control, file and data management, memory management, and communication control and related services, all in accordance with known techniques.

System memory may be any of a variety of known or future memory storage devices. Examples include any commonly available random access memory (RAM), magnetic medium such as a resident hard disk or tape, an optical medium such as a read and write compact disc, or other memory storage device. A memory storage device may be any of a variety of known or future devices, including a compact disk drive, a tape drive, a removable hard disk drive, or a diskette drive. Such types of memory storage device typically read from, and/or write to, a program storage medium (not shown) such as, respectively, a compact disk, magnetic tape, removable hard disk, or floppy diskette. Any of these program storage media, or others now in use or that may later be developed, may be considered a computer program product. As will be appreciated, these program storage media typically store a computer software program and/or data. Computer software programs, also called computer control logic, typically are stored in system memory and/or the program storage device used in conjunction with a memory storage device.

In some embodiments, a computer program product is described comprising a computer usable medium having control logic (computer software program, including program code) stored therein. The control logic, when executed by processor, causes the processor to perform functions described herein. In other embodiments, some functions are implemented primarily in hardware using, for example, a hardware state machine. Implementation of the hardware state machine so as to perform the functions described herein will be apparent to those skilled in the relevant arts.

Input-output controllers may include any of a variety of known devices for accepting and processing information from a user, whether a human or a machine, whether local or remote. Such devices include, for example, modem cards, network interface cards, sound cards, or other types of controllers for any of a variety of known input devices. Output controllers of input-output controllers could include controllers for any of a variety of known display devices for presenting information to a user, whether a human or a machine, whether local or remote. If one of the display devices provides visual information, this information typically may be logically and/or physically organized as an array of picture elements, sometimes referred to as pixels. A graphical user interface (GUI) controller may comprise any of a variety of known or future software programs for providing graphical input and output interfaces between a computer and user, and for processing user inputs. In the illustrated embodiment, the fictional elements of a computer communicate with each other via a system bus. Some of these communications may be accomplished in alternative embodiments using network or other types of remote communications.

As will be evident to those skilled in the relevant art, applications, if implemented in software, may be loaded into system memory and/or a memory storage device through one of the input devices. All or portions of applications may also reside in a read-only memory or similar device of memory storage device, such devices not requiring that applications first be loaded through input devices. It will be understood by those skilled in the relevant art that applications, or portions thereof, may be loaded by processor in a known manner into system memory, or cache memory, or both, as advantageous for execution.

A resequencing array is an array of probes comprising at least a first set of probes that is complementary to a reference sequence (or regions of interest therein). Typically, the probes tile the reference sequence. Tiling means that the probe set contains overlapping probes which are complementary to and span a region of interest in the reference sequence. For example, a probe set might contain a ladder of probes, each of which differs from its predecessor in the omission of a 5′ base and the acquisition of an additional 3′ base. The probes in a probe set may or may not be the same length. The number of probes can vary widely from about 1000, 10,000 or 100,000, to 10,000, 100,000, 1,000,000, 1,300,000 or 2,500,000. Typically, the arrays do not contain every possible probe sequence of a given length.

Often resequencing arrays have four probe sets, as described in WO 95/11995. The first probe set comprises a plurality of probes exhibiting perfect complimentarily with a reference sequence, as described above. Each probe in the first probe set has an interrogation position that corresponds to a nucleotide in the reference sequence. That is, the interrogation position is aligned with the corresponding nucleotide in the reference sequence, when the probe and reference sequence are aligned to maximize complimentarily between the two. For each probe in the first set, there are three corresponding probes from three additional probe sets. Thus, there are four probes corresponding to each nucleotide in the reference sequence-representing three possible variants at that position. The probes from the three additional probe sets are identical to the corresponding probe from the first probe set except at the interrogation position, which occurs in the same position in each of the four corresponding probes from the four probe sets, and is occupied by a different nucleotide in the four probe sets.

A substrate comprising the four probe sets is hybridized to a labelled target sequence, which shows substantial sequence similarity with the reference sequence, but which may differ due to e.g., species variations or polymorphism. The amount of label bound to probes is measured. Analysis of the pattern of label revealed the nature and position of differences between the target and reference sequence. For example, comparison of the intensities of four corresponding probes reveals the identity of a corresponding nucleotide in the target sequences aligned with the interrogation position of the probes. The corresponding nucleotide is the complement of the nucleotide occupying the interrogation position of the probe showing the highest intensity. The comparison can be performed between successive columns of four corresponding probes to determine the identity of successive nucleotides in the target sequence.

In many instances of comparing four corresponding probes, one of the four probes clearly has a significantly higher signal than the other three, and the identity of the base in the target sequence aligned with the interrogation position of the probes can be called with substantial certainty. However, in some instances, two or more probes may show similar but not identical signals. This may represent a heterozygote, e.g. two different alleles are present.

Methods for analyzing data from resequencing arrays and other methods relating to the use of resequencing arrays may be found, for example, in provisional U.S. patent application Ser. No. 10/028,482 which is incorporated herein by reference in its entirety and in Cutler et al. Genome Research 11(11):1913-1925, November 2001, which is incorporated herein by reference in its entirety. Additional methods for generating custom arrays may be found in U.S. patent application 10/063,559 and U.S. provisional applications 60/301,298, 60/222,522 and 60/376,033 each of which are incorporated by reference herein in their entireties.

(C) Custom Design of Resequencing Array and Assay.

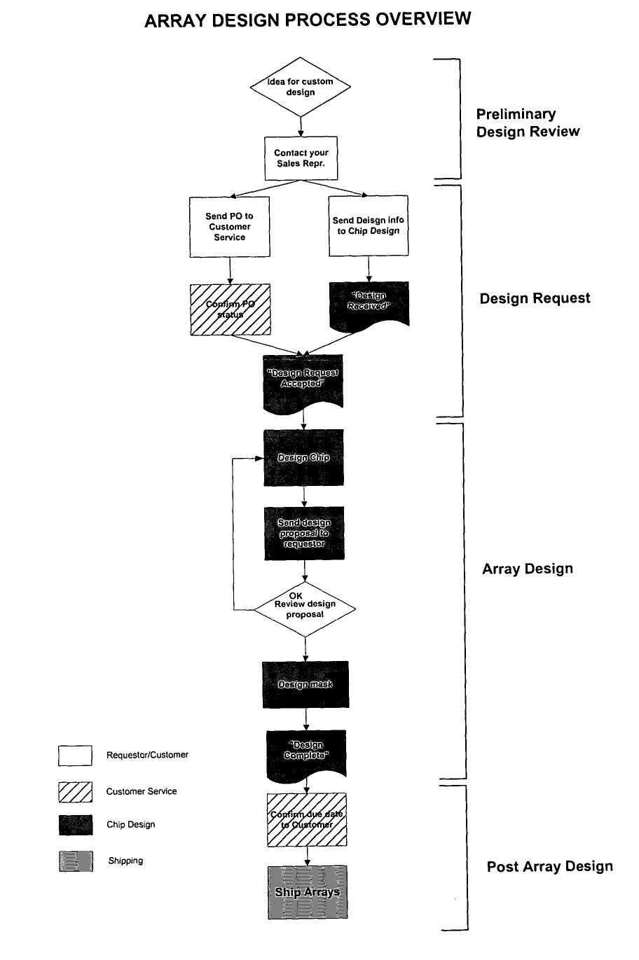

In one embodiment the invention provides a method for designing a custom array by working with at least one user to select a sequence of interest, design primers and an assay to allow analysis of the sequence in a variety of samples, and to design and produce an array to interrogate the sequence of interest. In a preferred embodiment the design process consists of five major steps: 1. ) Preliminary Design Review, 2. ) Sequence Selection and Primer Design, 3. )Design Request, 4. ) Array Design and 5. ) Post Array Design. FIG. 1 shows a flow chart of a preferred embodiment of the design process (sequence selection is omitted). The requestor (customer/user) identifies a need for a custom designed array and contacts the provider during the preliminary design review. The user has identified a sequence or sequences for the array and communicates that information to a designated chip design person or persons associated with the provider. The user sends a purchase order (PO) to the provider. Provider may confirm the PO status using a customer service representative and chip design may confirm receipt of the sequence information. Provider then communicates to customer that the design request has been accepted and may provide the customer with an estimate for completion of the design and/or delivery of the completed arrays. Chip design will then design the chip and send the design proposal to the customer. The customer will then review the design and approve, request modification or reject the design. Modifications or rejections may be returned to the chip design group to revise the design. After design acceptance masks are designed and made. A due date may be communicated to the customer. The arrays are then synthesized and shipped to the customer.

In a preferred embodiment arrays are designed to detect polymorphisms in the selected sequence or sequences by resequencing the sequence from a plurality of individuals or sources. The polymorphisms may be novel polymorphisms or polymorphisms that are known to occur in one or more population. See also U.S. Patent Publication No. 20030120432 which is incorporated herein by reference in its entirety.

1. Preliminary Design Review

To begin the process for ordering a custom resequencing array, a user should contact an appropriate account manager at the providing organization. The account manager may then review the request with a Field Application Specialist (FAS) and one or more members of a Custom Array Design Team.

In some embodiments the design request is straight forward, and the account manager and FAS will work with the user to prepare sequence submission documents and submit a Purchase Order. If the design idea requires modification to the design standard, a technical group may be made available to help. In some embodiments, a meeting or conference call may be arranged with the Custom Array Design Team. The initial review process is designed to provide the user with the array design that meets the user's needs.

2. Sequence and Primer Selection

In many embodiments the first step to designing a custom array is identifying the sequence(s) of interest. Once genomic region(s) are selected for analysis, sequence may be converted to the proper format and quality checked (see Section 0). Next, PCR primers may be designed to amplify the region(s) and amplicons tested for adequate target amplification. In many embodiments a step is included to ensure that the sample has adequate biological material to detect specific sequences before submitting a design for manufacture.

3. Design Request

Next, sequence is submitted to the provider by filling out a form, for example, a Design Request Form, and sending the formatted sequence and instruction files to, for example, a Chip Design team. Once the design information is received, the Chip Design team may send the user a communication indicating that the design has been received. In some embodiments of the invention messages are in the form of an e-mail message with a subject line that describes the array and project.

In some embodiments the design process begins after the provider receives a purchase order and confirms that the purchase order is complete. Once everything is complete, a confirmation message may be sent to the user, for example, to the user's Purchasing Agent.

When both the completed Purchase Order and the design information have been received, the Chip Design Group will send the user a communication, for example, a “Design Request Accepted” message. This message signifies that array design will begin on the user's custom array.

4. Array Design

In some embodiments the array design process begins by assigning a specific chip designer to the user's design. This individual is the contact person throughout the design process. If the designer has questions, he/she will send the user a “Design Clarification” message.

Upon completing the mask design, the provider will send the user a “Design Complete” message. This message signifies that the chip design is complete and the arrays will be manufactured.

In many embodiments the provider sends a series of communications to update the user on the status of the array design as it moves through the design process.

5. Post Array Design

After the arrays are manufactured and quality control tested, they are shipped to the user along with Library Files containing information specific to the design. Technical questions should be directed to the field application specialists, while all other questions may be directed to an account manager.

Resequencing Design Standards and Considerations

Sequence Selection

In some embodiments the method allows researchers to design custom arrays containing unique content. The sequence selected may first be identified by using a genotyping method that identifies a large region of interest, for example a linkage analysis study or an association study. Sequence of interest may be downloaded from any number of public or proprietary databases and converted to an acceptable format, for example, FastA format.

In some embodiments about 30,000 bases may be resequenced on an array with feature size of 25×20 microns. In another embodiment about 10,000 bases may be resequenced. Smaller feature sizes may be used to resequence larger regions of sequence.

Repetitive elements and internal duplications may lead to cross-hybridization and in some embodiments they are removed prior to array design. In some embodiments repeats and other repetitive regions or large duplications are removed by a computer program. Methods to remove repeats include, for example,the use of RepeatMasker shareware available on the internet. To remove other types of repetitive regions and large duplications MiroPeats, for example, may be used, this is also available on the internet.

Highly homologous sequences may also lead to cross-hybridization and compromised data quality for that specific sequence. In some embodiments a homology check is run on both your amplified and tiled sequences prior to submission. Highly homologous regions may be tiled on separate arrays.

In some embodiments any ambiguous sequence (N's) submitted is randomly replaced with A,G,C, or T. In some embodiments additional or alternative sources of sequence may be consulted to manually edit the ambiguous bases.

In some embodiments primer selection and validation are used to ensure the array contains primarily sequence for which adequate biological material can be obtained. Primers may be designed external to the sequence interrogated on the array, and they do not need to be adjacent to the first base sequenced. A homology search may be run between the sequence of the amplified fragments and the sequence tiled on the array in order to limit cross-hybridization between similar sequences. In a preferred embodiment the amplified sequence is larger than the sequence interrogated on the array.

In one embodiment user may access a website that provides access to design tools for use by the user in preparing a sequence for submission in a design request for a custom resequencing array. The website may be password protected. Additional information such as a list of primers may also be available on a website. For a description of a web portal for custom design of an array see U.S. Patent Publication No. 20030097222.

In some embodiments resequencing arrays will have standard manufacturing and hybridization controls tiled on the arrays. In some embodiments resequencing arrays will also contain DNA analysis control sequences to test for amplification from a synthetic construct. In another embodiment users may select controls to be tiled on the array.

In some embodiments additional controls may be tiled on the array(s). In some embodiments these sequences are included in the array capacity calculation and added to the sequence submission file. For example, for Human DNA, the p53 gene may be tiled in some embodiments to validate amplification from sample DNA.

Array Format and Size

In some embodiments the provider requires the user to order a minimum number of arrays per synthesis for the corresponding array formats. Custom products in some embodiments are ordered in full manufacturing lots. For example, for standard size the minimum order may be 80±10 and for the Midi size the minimum order may be 90±5.

In some embodiments probes for both the forward and reverse strands are tiled on the array, the feature size is less than 500 square microns, less than 400 square microns or less than 100 square microns. In some embodiments the feature size is 25×20 micron, in other embodiments the feature size ranges from 5, 10, 11, 15, 18 or 20 to 25×5, 10,11, 15, 18 or 20 to 10, 20 or 25 microns. In a preferred embodiment the feature size is 11×11, microns. In other embodiments the feature size is 18×18 or 8×8 microns. The smaller the feature size the larger the number of probes and the larger the amount of sequence that can be interrogated. For example when the feature size is 11×11 approximately 1.3 million probes features may be present on a single array. Where the feature size is 8×8 microns approximately 2.5 million features may be present on a single array. In a preferred embodiment the probe length is 25 bases but the probe length may be about less than 100, 50 or 20 bases. The design may be such that a single array is used or in some embodiments larger sequences may be interrogated by combining two or more arrays.

In some embodiments a set of Library Files is generated for each custom resequencing design. The Library Files include a list of all the probes tiled on the array and the location of each probe. In addition, the Library Files also include the correct parameters for a fluidics station protocol and the standard analysis parameters recommended for the computer software. An installation program may also be provided for each design, which loads the Library Files into a computer system.

In one embodiment array designs are prepared in advance for selected regions of a genome. Regions selected may be those known to contain genes in, for example, the human genome or a genome of a pathogen. In one embodiment array designs are generated for known genes of interest. Regions of the genome that contain genes or features of interest may be the subject of array designs. Users may then order the arrays without providing the sequence and without analyzing the sequence to, for example, remove repetitive sequences and eliminate ambiguous sequences. In one embodiment the provider performs the steps needed to prepare a finalized sequence for array design.

In many embodiments genomic DNA is amplified using PCR prior to hybridization to the custom resequencing array and in some embodiments the provider may assist the user in selection of primers for PCR. In some embodiments the provider may preselect PCR primers to be used with predesigned arrays. For example, the provider may select a sequence that may be of interest to one or more users, for example, the SARS genome, and prepare a design for the selected genome and also select sequences to be used as primers to amplify regions of the selected sequence for analysis. The user may then simply order the design in the appropriate format. The provider may or may not have tested the primers to determine functionality.

In one embodiment the provider has designed arrays for large regions of a genome, for example, for an entire human chromosome or for the entire human genome or a large portion thereof. The user would then simply request an array or arrays that contain the region that the user is interested in. The user may, for example, request arrays to resequence an entire chromosome of interest. The provider may also provide the customer with access to primer sequences that amplify the sequence interrogated by the pre-designed array(s).

Method for Submitting a Design Request

In some embodiments users make a formal commitment to purchase a minimum number of arrays prior to design of the array. In one embodiment the formal commitment is in the form of a purchase order. In some embodiments a purchase order for a custom resequencing array includes a design fee and an order for a first lot(s) of arrays.

In some embodiments a user may submit a form specially designed for requesting a custom array. The form may provide the provider with contact information and design parameters for the array design. The form with blank fields for the information is in some embodiments available from the internet or in another available electronic media.

User information fields in the form provide the contact information to notify the design requester of the status of the design. In some embodiments the provider may also contact the requestor for questions/clarifications about the design. Optional fields for information that may be helpful to the provider may also be included on the form.

In some embodiments a cross-hybridization threshold is selected to be an integer between 1 and 100. This threshold is used to determine if there is too much cross-hybridization within a design such that it should be split into a new design. If the percentage of probes that cross-hybridize with other sequence(s) over the total number of probes is greater than or equal to the cross-hybridization threshold, then the cross-hybridizing sequences may be put on two different designs in some embodiments. In preferred embodiments the cross-hybridization threshold is 10 or less.

In some embodiments an array may be designed to interrogate sequence from more than one species of organism. In some embodiments species-specific controls are tiled on the array. In some embodiments a Sequence File(s) is generated, containing all the sequences from which probes will be selected. In some embodiments 12 bases are added at the start and end of each fragment.

In preferred embodiments the sequence file is in an appropriate computer readable form. In some embodiments the sequence file is in the FASTA format. In some embodiments each raw sequence is preceded by a definition line. One of skill in the art will appreciate that any other appropriate format may be used for the sequence file. In some embodiments the sequence file has one or more of the following characteristics: the definition line begins with a sign, and is followed immediately with a name for the sequence; A “>” precedes the sequence name, the sequence name is unique; the sequence name corresponds to the value in the “name” column in an instruction file; all names defined in the instruction file exist in the sequence file, and vice versa; the sequence name in the design and pruning sequence files may be up to 20 characters that are alphanumeric or special characters: “+”, “@”, “$”, “%”, “\hat{ }”, “&”, “(”, “)”, “.”, “−”, “₁₃”, “=”, “#”, “˜”; in some embodiments some characters and some sequences of specific characters are not allowed by the provider because they have been used as identifiers by the provider. The user may include a comment following the sequence name which may be ignored during the design of the array. Any annotations the user would like to include in the Library Files may be included in the instruction file. In some embodiments there are no blank lines between sequences. For additional description see the CustomSeq Custom Resequencing Array Design Guide (2003) which is incorporated by reference and is available from Affymetrix, Inc., Santa Clara, Calif. and also available at the Affymetrix website, Affymetrix.com. An example is shown below with the sequence in FastA format:

>AA618977 any comments here are ignored
gttttgtctttggttaaagtaccttttgcatcatgattctttgagatgtttagattatttctagttcccgaatggcttatgattttcattgattca
tagcaagtttgtgcatagataagttgttgtgtaaacatttgtagaaatcattgaggttgtgataaataattcgtatagtctgataactggtttatagct
tgattcttattgttgatgaaaaaaaa
>AA618981
agatcttacggactatctgatgaagatcttgactgagagaggttacagttttacgacgacagcggaacgtgagatagtccgagac
atcaaggagaaactgtgttatgttgccttggatttcgaacaggagatgggtacggcagcttcgagttcggcgttggagaagagttatgagctt
cctgatggtcaagtgattactattggtaacgagcggttaattctattatgaagtgtgacgtagatatccgtaaagatctgtacccaacacagtatt
gtc
>ABCD
agatcttacggactatctgatgaagatcttgactgagagaggttacagttttacgacgacagcggaacgtgagatagtccgagac
atcaaggagaaactgtgttatgttg

In some embodiments the instruction file provides a tabular summary of the start and end position for each fragment tiled on the array. This information is useful for accurate array design, as well as being a component of the quality control process. This file can easily be created as a simple word document with a separate entry for each sequence in the Sequence File. An example is shown below:

| | | | | | | |
|---|---|---|---|---|---|---|
| Name | Alias | Start | End | StartSeq | EndSeq | Design |

| | | | | | | |
|---|---|---|---|---|---|---|
| AK097958p53FLa | p53exon1 | 1 | 70 | ACGTATGA | AGCATGTA | 1 |
| AK097958p53FLb | p53exon3-4 | 298 | 821 | AGTCGTAT | ATCGTAGT | 1 |
| AK097958p53FLb | p53exon5 | 809 | 1924 | ACGTAGTC | GCTGCTGA | 1 |

In some embodiments the “name” is the unique designation for each gene or sequence represented on the array. A “name” may be provided for each sequence described in the Design Sequence File.

The Accession Number in GenBank or the unique sequence ID from a public domain or proprietary database may be used, for example. Utilization of standard accession numbers facilitates linkage to annotations during data analysis.

In some embodiments the “alias” is the unique designation used for the fragment name. When tiling multiple non-contiguous fragments for the same gene or sequence, an ‘Alias” may be used to differentiate between them. The contents of the “name” field may also be used as the alias name.

In some embodiments the Start designates the first base in the fragment. This refers to the base at the beginning of the probe, and not the first position of interrogation. The first base sequenced is position 13 in the sequence file while position 1 is the first base tiled. An example is shown below.

| Base | ACGTTGCATGTGTTATAGCTAGTCATGCATCGTGC . . . |
|---|---|
| Position | 1 13 |

In some embodiments the “end” designates the last base in the sequence fragment. This refers to the absolute position of the end of the probe, which is 12 bases after the last base of interest. For example, if your last base of interest is at base 2000 (last base sequenced), then the “end” value may be specified to be base 201 (last base tiled). An example is shown below:

| Base | . . . CATGTGTTATAGCTAGTCATGCATCGTGC |
|---|---|
| Position | 2000 2012 |

If multiple probe selection regions from the same sequence are specified, the “end” (last possible probe) from the first region and the “start” (first possible probe) from the next region do not overlap by more than 24 bases in some embodiments. An example is shown below:

| Name | Alias | Start | End |
|---|---|---|---|
| AK097958p53FL | p53exon1 | 1 | 70 |
| AK0979S8p53FL | p53exon2-5 | 298 | 821 |
| AK097958p53FL | p53exon2-5 | 809 | 1924 |

The “StartSeq” is used for quality control of the sequence file in some embodiments. It includes the first 8 bases of the sequence file. This information allows a cross-check of the sequences and the fidelity of the sequence file.

The “EndSeq” is used also for quality control of the sequence file in some embodiments. It includes the last 8 bases of the sequence file. This information allows a cross-check of the sequences and the fidelity of the sequence file.

An example of an instruction file for a simple design is shown below. The first base of interrogation starts at base 13 for sequence A, and base 1012 for sequence B. The last base of interrogation for sequence A is at base 787, and base 1987 for sequence B.

| | | | | | |
|---|---|---|---|---|---|
| name | alias | start | end | startSeq | endSeq |

| | | | | | |
|---|---|---|---|---|---|
| AK097958.a | p53exon1 | 1 | 70 | ACCG | CCGT |
| AK097958.b | p53exon2 | 500 | 800 | GGGA | TAAA |
| AK097958.b | p53exon5 | 1500 | 2000 | GGGA | TAAA |

If sequences A and B are very similar to each other, in some embodiments tiling to different designs is specified.

| | | | | | |
|---|---|---|---|---|---|
| name | start | end | startSeq | endSeq | design |

| | | | | | |
|---|---|---|---|---|---|
| A | 1 | 70 | ACCG | CCGT | 1 |
| B | 100 | 200 | GGGA | TAAA | 2 |

In some embodiments a custom designed resequencing array is part of an integrated approach to analysis of a biological system wherein each step is designed to allow the researcher to focus on a progressively smaller region of a genome. For example, a researcher may first scan the entire genome by, for example, genotyping a subset of SNPs that are spaced throughout the genome. For this application researchers may use, for example, an Affymetrix GeneChips® Mapping Array such as the Mapping 10 K Array available from Affymetrix, Inc., Santa Clara, Calif. The Mapping 10 K Array genotypes more than 10,000 human SNPs and is described in provisional U.S. patent application No. 60/470,475 filed May 14, 2003, which is incorporated herein by reference in its entirety. The mapping array may be used to identify regions of the genome that are of interest for further study, for example, a region that is associated with a particular phenotype may be identified. The identified region or regions of interest may then be the subject of further study wherein a smaller amount of the genome is analyzed at higher resolution so that, for example, a higher density of SNPs may be analyzed. For example, if a region of approximately 400 Kb is identified as being associated with a particular phenotype assays may be derived for analysis of some or all of the SNPs in that region. SNPS are predicted to be present on average approximately every 1000 bases so a 400 Kb region would be expected to have about 400 SNPs. In some embodiments only a subset of the SNPs in a region have been identified or are publicly available. Having identified a region of interest a researcher may analyze the genotype of some or all of the SNPs in that region to do a fine mapping of that region. Any genotyping method known in the art may be used, see for example, Syvanen, Nat. Rev. Genet. 2: 930-942 (2001), Twyman and Primrose, Pharmacogenomics 4:67-79 (2003) and Jenkins and Gibson, Comp. Funct. Genom. 3:57-66 (2001), each of which is incorporated herein by reference in its entirety. In a preferred embodiment the assay selected for fine mapping is locus specific for each SNP to be genotyped and a GenFlex Tag Array, available from Affymetrix, Inc. is used. For a description of Tag Array technology, see U.S. Pat. No. 6,458,530 which is incorporated herein by reference in its entirety.

The fine mapping assay methods may, for example, identify a smaller region(s) that is associated with a particular phenotype, for example, a region that is less than 30 Kb may be identified. In a preferred embodiment the region(s) identified by fine mapping are used to design a custom resequencing array. The array has the region of interest tiled for resequencing and may be used to identify polymorphisms in the region of interest. This may be used, for example, to genotype known polymorphisms, to discover new polymorphisms or to determine frequency of polymorphic alleles in a population. In one embodiment the provider has predesigned an array for analysis of the identified region.

In another embodiment the provider also provides the user with access to the sequence of primers that may be used to perform long range PCR in the region of interest. In a preferred embodiment the primers have been tested and are known to function in long range PCR. In a preferred embodiment the provider has a database of primer sequences that will amplify a majority of the genome of interest. For example, the provider may have a database of primer pairs that may be used to amplify more than 80%, or more than 90% of the human genome so that when a user identifies a region of the genome that the user would like to resequence the provider may provide the user with a custom resequencing array, primers to perform long range PCR to amplify that region and a protocol for performing the amplification. The user may submit a purchase order for the custom resequencing array, or otherwise commit to purchase the array, and the provider may provide the user with access to the sequences of primers that For a description of iterative resequencing see U.S. Patent Publication No. 20020025520, WO 95/11995, and EP 717,113 which are each incorporated herein by reference in their entireties.

In many embodiments the custom resequencing arrays are used in a high throughput manner. For a description of high throughput screening methods see PCT Publication No. 03/060526 and U.S. Patent Publication No. 20030124539 which are each incorporated herein by reference in their entireties.

In a preferred embodiment nucleic acids are amplified by PCR prior to hybridization to the resequencing array disclosed above. The array may be designed to interrogate the amplified fragments for polymorphisms. The nucleic acids may be amplified by long range PCR using primers that are designed to amplify specific regions of a nucleic acid. The array is tiled with probes to those regions. Probes are included on the array to detect variation in the sequence. In some embodiments the amplification and hybridization methods may be combined with a computer method for detection of variation in a sequence.

Various strategies may be employed to amplify the target sequences from Genomic DNA, as illustrated in FIG. 1. Long-range PCR amplification may be used when appropriate to reduce assay cost and complexity. In one embodiment the invention contemplates use of long range PCR, as outlined in FIG. 1. In another embodiment traditional short PCR amplification is used, and it may be desirable to begin with Step 2 in the protocol, “Quantitation and Pooling of PCR Amplicons”.

### EXAMPLE 1

### Resequencing Assay

The following provides a nonlimiting example of how a user may perform a resequencing assay using an array designed according to the methods above. In a preferred embodiment the provider supplies the user with a protocol to assist the user in performing the assay. Throughout the example suggested reagents and equipment are provided, however, one of skill in the art will appreciate that substitutions from other vendors may be made. The steps of the assay are as follows: 1. ) long range PCR, 2. ) DNA quantitation, 3. ) Fragmentation and labeling, 4. ) Controls, 5. ) hybridization, 6. ) wash and staining, 7. ) scanning.

For long range PCR the following reagents and equipment may be used: LA PCR Kit Ver. 2.1, TakaRa Bio Inc. (p/n RR013A, also available from Fisher p/n TAKRR013A) containing: 10×LA PCR Buffer II (Mg2+): 1 mL/vial dNTP Mixture: 800 uL/vial and TaKaRa LA Taq: 5 units/uL; AccuGENE Molecular Biology Grade water and Ambion 1×TE, pH 8. 99.9% DMSO Sigma (p/n D-8418), PCR primers and a thermocycler.

For DNA quantitation Picogreen Molecular Probes (p/n P-7589); QIAquick PCR clean up kit, Quiagen spin columns (p/n 28104) or 96 well plate (p/n 28181) and a Fluorescent reader may be used.

For fragmentation and labeling Terminal Transferase (New England Biolabs p/n M0252S), Bio-N6-ddATP (Perkin Elmer Life sciences p/n NEL508), 10×OnePhorAll Buffer, (Amersham Life Sciences P/N 27-0901-02), NOVEX Pre-cast gels: 20% TBE Gel (Catalog # EC63155), 25 BP ladder (Invitrogen p/n 10597-011), SYBR Gold Mol Probes (p/n S11494) and a thermocycler may be used.

Controls may include: Affymetrix Custom-Seq Control kit containing Fragmentation Reagent, Oligo B2 and Tag IQ-EX control system.

For hybridization 5 M Tetramethylammonium chloride solution (TMAC) Sigma (p/n T3411), Triton X-100 Sigma (p/n X-100-RS), Acetylated Bovine Serum Albumin (BSA) solution Invitrogen conc. 50 ug/ul (p/n 15561-020) and Herring sperm DNA (Promega corp p/n D1811) may be used along with a hybridization oven.

Washing and staining may be done using SAPE Molecular Probes (p/n S-866), Anti-streptavidin antibody (goat), biotinylated, Vector Labs (p/n BA-0500), Goat IgG Reagent Grade Sigma (p/n 15256), 20×SSPE (3 M NaCl, 0.2 M NaH₂PO₄, 0.2MEDTA) AccuGENE, (p/n 51214) and an Affymetrix Fluidics station.

The Affymetrix Tag IQ-EX control set may be used as a control. The purpose of this control set is two fold; first to act as a control for PCR reactions, second to act as hybridization controls. When conducting the PCR reactions in some embodiments at least one positive control and one negative control reaction are carried out on every 96 well plate

Positive control reactions may use the appropriate primer pair and template from the Tag IQ-EX set, as described. The appropriate sized product may be observed when the reaction is run on the TBE gel. In some embodiments negative control reactions are used and may consist of a set of user primers with no template. In negative control reactions o product may be seen on the TBE gel.

In some embodiments hybridization controls may be added to every sample prior to hybridization of the sample on an array. The hybridization controls may be prepared in a separate PCR from the product PCR. Preparation instructions are provided below. In some embodiments a control PCR is included as a control for Long Range PCR Reactions (7.5 kb fragment). In some embodiments one or more primer pairs are designed to provide products of an expected size. For example, the primers in the following protocol may be used when the average expected product size is greater than 5 kb.

In some embodiments the following control mix may be prepared: 5 μl Tag IQEX template, 3 μl Forward Primer 7.5 kb, 3 μl, Reverse Primer LR and 29 μl water. Add 40 μl of the control mix to the reaction vessels. If using 96 well plates in some embodiments it is recommended that at least 1 control per plate be included. If using strips of tubes you may add 1 control mix to every strip. Add 60 μl of the PCR master mix to each control mix (as described in the Long range PCR protocol). Resolve the control product on a 1% agarose gel (as described in the Long range PCR protocol). A 7.5 kb band may be observed. Pool remaining contents of the control wells together and clean up the pool using the Qiagen QIAquick purification kit. Quantify the pool after clean up using the methods described in the Pooling protocol. Fragment and label the control pool. Add 0.26 μg of the labeled mix to each array. A single PCR control reaction may provide sufficient material for around 4-5 Arrays. If insufficient control PCR reactions have been preformed, Spike in Control Material can be used as substituted.

By varying the primers used the Tag IQEX template may be used as a control for medium range PCR (3.5 kb band) or for short range PCR (1.0 kb band).

Step 1: Long Range PCR Protocol

Reagents and Materials: LA PCR Kit Ver. 2.1, TakaRa Bio Inc. ((p/n # RR013A, also available from Fisher p/n TAKRR013A) (containing: 10×LA PCR Buffer II (Mg2+): 1 mL/vial, dNTP Mixture: 800 uL/vialand TaKaRa LA Taq: 5 units/uL ), AccuGENE Molecular Biology Grade water, Ambion 1×TE, pH 8 (p/n 9849) diluted 10-fold in water to give 0.1×TE, 99.9% DMSO Sigma (p/n D-8418) and Affymetrix CustomSeq™ Control kit (p/n 900XXC).

Oligonucleotides may be ordered from a vendor or synthesized. Standard salt-free purification is sufficient. Primers may be tested prior to finalizing the array design in order to ensure robust amplification. First re-suspend oligonucleotides in 0.1×TE to 100 μM. The stock can then be stored at −20° C. Combine the following to create a primer pair stock: 1001 μM Forward primer (100 μM, 100 μl Reverse primer (100 μM), and 800 μl 0.1×TE. The final concentration of the diluted stock may be 10 μM for each primer. 6 μl of the resulting primer pair stock may be aliquoted into 96 well plates and stored at −20C. until required.

In a preferred embodiment the genomic DNA used in this assay is of high quality. In some embodiments particular attention is paid to ensuring that the DNA is free from any PCR inhibitors. The concentration of the Genomic DNA may be measured by absorbance spectroscopy or by using a reagent such as Picogreen®. The DNA may be diluted to 5 ng/μl in molecular biology grade water and stored at −20° C. In some embodiments the aliquots of each genomic DNA are stored at −20° C. as 40 μl aliquots in singe wells of 96 well plates. The DNAs may be added to the PCR mixes quickly using a multi-channel pipettor. In some embodiments the frozen aliquots are not subjected to more that 3 freeze/thaw cycles.

In many embodiments the Pre-PCR Clean room/workspace is free from template DNA and PCR product. In some embodiments gowns and gloves are used to prevent PCR carryover. In some embodiments the Long Range PCR Reaction is conducted in 96 well plates. A recommend layout of a 96 well plate is to keep the primer pairs together in rows so that the genomic DNA can be added using a multi-channel pipettor. In some embodiments downstream handling conditions are taken into consideration, for example, factors such as whether or not a robot will be used to process samples and if so whether the samples will be processed more efficiently in columns than in rows are taken into consideration.

To wells of a 96 well plate containing the PCR primers 14 μl of molecular biology grade water may be added. Each well may now contain: 6 μl each primer pair stock and 14 μl molecular biology grade water. The plate may be moved to the PCR staging Room. In many embodiments the PCR Staging room/workspace should be free from any PCR product. Gowns and gloves may be used to prevent PCR carryover. In the PCR staging room add 20 μl of genomic DNA to each well primer pair mix and water. The total volume of each well should now be 40 μl . Prepare the PCR master mix and keep it on ice to prevent primer degradation from the proof reading activity of the polymerase. The PCR master mix is 33.0 μl water, 16 μl 2.5 mM dNTPs (final concentration of 400 μM), 10 μl 10×LA PCR buffer (Mg²⁺) (final concentration of 1×), 1 μl LA Taq enzyme (final concentration of 5 U/100 μl) for a total of 60 μl. In some embodiments DMSO may be used for problematic PCRs. In others it is unnecessary and even inhibitory. For a high GC template in some embodiments DMSO may be used to a final concentration of up to 5.0% and the volume of water in the reaction reduced accordingly.

For PCR the block may be preheated to 94° C. To minimize degradation of the primers by the polymerase, thermocycling may begin as soon as possible after adding the PCR mix to the DNA/primers. In some embodiments a co-worker in the main lab may preheat the PCR block. 60 μl of the PCR master mix is added to each well. Keep the PCR master mix and DNA-primer plate cold till ready to cycle to avoid primer degradation by proof reading enzyme. The plates may be sealed the plates using for example the MJ Research Microseal “A”. For each reaction final Primer concentration may be 600 nM (each primer) and final DNA template concentration may be 100 ng/1001 μl.

In some embodiments the Main Lab is assumed to have higher levels of airborne contamination with PCR product and template than the PCR clean room or PCR staging area. After entering the main lab it is unadvisable to re-enter either the Pre-PCR Clean Room or the PCR Staging area. In the main lab place the PCR reaction plates in the pre-heated thermocycler and run the following 1X program: 94 ° C. for 1 cycle at 2 minutes, 94 ° C. for 15 seconds, 68° C. for 1 minute /kb for 30 cycles, 68° C. for 5 minutes +1 minute/kb and finally hold at 4 ° C. following thermal cyclers have been successfully used in this assay, MJ Research Tetrads and PE 9700, however, other thermal cyclers may be used. The success of the PCR may be verified by running 4 μl of each reaction on a 1% TBE gel.

Step 2: Pooling and Quantification of PCR Amplicons

The efficiency of a PCR reaction can vary between samples. Assay performance may be compromised if amplicon concentration varies by more than 2 fold. Therefore to achieve the maximum amount of sequence from a single hybridization, in some embodiments similar quantities of each PCR reaction are applied to the array. First, each PCR reaction may be quantified, and then equi-molar amount of each PCR product may be pooled, prior to the fragmentation and labeling of the product.

Quantitation and Pooling: Examples of methods to quantify the products from the PCR reaction include but are not limited to: using a double stranded DNA specific Dye (e.g Picogreen®) and using absorption spectrophotometry. The advantage of the first method is that primers and dNTPs do not need to be removed from the mixture prior to quantitation, so pooling equimolar quantities of each amplicon can be done before purification, and thus fewer purifications steps are needed. If the second method is used primers and dNTPs should be removed from the mixture prior to quantitation. The Qiagen Qiaquick kit can be used to clean up samples prior to quantitation.

Step 3: Fragmentation and Labeling Reaction

The fragmentation reaction is an enzyme reaction and is sensitive to time and temperature. In some embodiments it is important to observe strictly the conditions of this reaction as any changes in condition can lead to sub-optimal product.

Reagents and Materials that may be used include: Terminal Transferase New England Biolabs (p/n M0252S) 20 Units /ul; Bio-N6-ddATP Perkin Elmer Life sciences (p/n NEL508) 250 nmoles in 100 μl (1 mM); 10×OnePhorAll Buffer, Amersham Life sci (p/n 27-0901-02); Fragmentation Reagent from: Affymetrix Custom-Seq Control kit p/n (900XXC); 20% TBE Gel Invitrogen (p/n EC63155); 25 BP ladder Invitrogen (p/n 10597-011); SYBR Gold Mol Probes (p/n S11494); and Ambion 1×TE, pH 8 (p/n 9849)diluted 10-fold in water to give 0.1×TE.

For the fragmentation reaction pool the PCR products from each DNA and make up the volume to 35 μl with Qiagen Buffer EB. Place the tubes on ice. Determine the quantity of Affymetrix Fragmentation reagent to use based on using 0.15U DNAse/μg DNA. Prepare the Fragmentation Cocktail by mixing 5 μl 10×OnePhorAll buffer, 5 μl10 mM Tris, pH7.8, and 4 μl Fragmentation Reagent in a total of 15 μl. Add 15 μl of the Fragmentation Cocktail to each 35 μl pool and store on ice. The total volume may now be 50 μl Place the tubes in a thermal cycler pre-heated at 37° C. and run the following program: 15 minutes at 37° C., 15 minutes at 95° C. and hold at 4° C. In some embodiments if sufficient volume is available from pooling, run 15 μl of the reactions along with 25 bp ladder on a 20% TBE PAGE gel to ensure that the fragmentation is complete. The gel, may be stained with SYBR Gold diluted:10,000 in TE and stain the gel for 30 minutes. The fragmented DNA should run between 50 and 200 bp approximately.

For labeling prepare the following cocktail: 1.5 μl/Rx Bio-N6-ddATP, 1.0 μl/Rx TdT 20U/μl. Add 2.5 μl of the labeling cocktail to each reaction. The total volume may now be 52.5 μl. Place the tubes in a thermal cycler pre-heated at 37° C. and run the following program: 37° C. for 90 minutes, 95° C. for 15 minutes and hold at 40° C.

Step 4: Target Hybridization

In some embodiments first prepare the pre-hyb and the hyb solutions. Then apply the pre-hyb solution to the arrays and start the pre-hybridization incubation. While the pre-hyb incubation is taking place you may commence denaturizing the samples.

Reagents and Materials that may be used include: Tetramethylammonium chloride solution (TMAC) Sigma (p/n T3411), Triton X-100 (Sigma p/n T2913) diluted to 1% in molecular biology grade water, Acetylated Bovine Serum Albumin (BSA) solution (Invitrogen p/n 15561-020), Herring sperm DNA (Promega Corp p/n D1811) and Oligo B2 from Affymetrix CustomSeq(TM) Control kit (p/n 900XXC).

Hybridization reaction: Prior to applying a sample to an array it may be useful to allow the arrays to normalize to room temperature completely. Specifically, if the rubber septa are not equilibrated to room temperature they may be prone to cracking which can lead to leaks. Prepare the following Hybridization Cocktail Master Mix: 3M TMAC, 10 mM Tris, pH7.8, 0.01% Triton X-100, 500 μg/ml Ac BSA, 100 μg/ml HS DNA, and 50 pM Oligo B2. Once prepared the Hybridization cocktail master mix can be stored at −20°0 C.

Prepare the following Pre-Hybridization Buffer3M TMAC, 10 mM Tris, pH7.8 and 0.01% Triton X-100. Once prepared the Pre-hybridization buffer can be stored at room temperature. Pre-Hybridize the array by filling the array with 200 μl of Pre-Hybridization Buffer. Place the Arrays in the Hybridization oven at 45° C. rotating at 60 RPM for 15 minutes. Add 167.5 μl of the Hybridization Cocktail Master Mix to the 52.5 μl fragmented and labeled DNA. Denature the Hybridization Cocktail by placing the tubes at 95° C. for 5 minutes. Equilibrate the Hybridization Cocktail by placing the tubes at 45° C. for 5 minutes. Vortex the tubes and briefly spin the tubes to collect any condensation from the side of the tubes. Remove the arrays from the Hybridization oven. Remove the Pre-Hybridization Buffer from the arrays and replace with 200 μl of hybridization cocktail. Return the arrays to the Hybridization oven for 16 hours at 45° C. rotating at 60 RPM. Take out the hybridization solution and save it at −20C. Completely fill the array with wash buffer A.

Step 5: Washing, Staining and Scanning

The arrays may be washed using the Affymetrix fluidics station and scanned using the Affymetrix scanner. The following reagents and materials may be used: SAPE (Molecular Probes p/n S-866), Anti-streptavidin antibody (goat), biotinylated, Vector Labs (p/n BA-0500), Goat IgG Reagent Grade Sigma (p/n 15256), and 20×SSPE (3M NaCi, 0.2M NaH₂PO₄, 0.2MEDTA) BioWhittaker, (p/n 16-010Y).

Before the 16 hours hybridization is finished prepare the following buffers and stains. Wash Buffers A and B can be stored at room temperature for a period of months. The 2 stain buffers, however, are preferably made fresh daily. Non-Stringent Wash Buffer A is 6 ×SSPE, and 0.01% Triton X-100 and may be filtered through a 0.2 μm filter and stored capped at room temperature. Stringent Wash Buffer B is 0.6×SSPE and 0.01% Triton X-100. SAPE Stain (Stains 1 & 3) may be made and dispenses into 600 μl aliquots that are protected from the light by storing in amber 1.5 ml tubes. SAPE stain is 6×SSPE, 0.01% Triton X-100, 2 mg/ml Ac BSA and 10 μg/ml SAPE. Antibody Stain (Stain 2) is mixed by pipetting up and down, instead of vortexing and may be dispensed into 600 μl aliquots. Antibody Stain is 6×SSPE, 0.01% Triton X-100, 2 mg/ml Ac BSA, 3 μg/ml Antibody and 100 μg/ml Goat IgG.

Wash and stain the arrays. Following the completion of the wash and stain process, ensure that no bubbles are trapped in the array. Remove bubbles by either replacing the array back in the fluidics station or by manually removing the bubbles with a pipette. Scan the Array using the Affymetrix scanner.

Step 6: Data Analysis

Following scanning of the arrays .CEL files are automatically created by the software. The .CEL files can then analyzed using the GeneChip DNA Analysis System (GDAS) software package.

In some embodiment the user should complete one or more of the following steps to obtain a custom resequencing array. Select sequence in FASTA format or another appropriate format. Remove repetitive elements. Edit ambiguous bases. Checked sequence homology. Determine if the number of bases will fit on desired format. Insert flanking regions (12 bases up and down stream of first base interrogated). Design and test primers. Submit a design request form. Select names for the Sequence and Instruction files. Submit the design to the provider. Submit a purchase order to the provider.

1. A computerized method for designing a resequencing array to resequence a user selected sequence comprising:
receiving a user design request comprising a user selected sequence;
producing an array design for resequencing said user selected sequence;
outputting said array design to said user;
receiving acceptance for said array design from said user;
outputting a file that is useful for controlling a nucleic acid synthesizer during the construction of an array comprising said array design;
synthesizing at least one copy of said array; and
providing said at least one copy of said array to said user.

2. The method of claim 1 wherein said nucleic acid synthesizer is maskless.

3. The method of claim 1 wherein said user design request comprises a sequence file.

4. The method of claim 1 wherein said user design request is received over the internet.

5. The method of claim 1 wherein the array further comprises user selected control probes.

6. A method for a provider of nucleic acid arrays to provide a user with an array for resequencing a user selected nucleic acid wherein the method comprises:
receiving a sequence computer file from said user wherein the sequence computer file comprises the sequence of the user selected nucleic acid;
preparing a design for a resequencing array for the selected sequence;
outputting said design into a design computer file;
providing said design computer file to said user;
receiving approval for said design from said user;
outputting an instruction computer file wherein said instruction computer file provides instructions to a nucleic acid synthesizer for synthesis of an array comprising said design;
synthesizing said array; and
providing said user with said array.

7. The method of claim 6 wherein after receiving from said user said sequence computer file the provider analyzes the sequence to remove repetitive sequences.

8. The method of claim 6 wherein said provider identifies ambiguous sequence within said user selected nucleic acid sequence by: obtaining analogous sequence from at least one other source; comparing the sequence from said file to said sequence from at least one other source; identifying bases that are different and removes ambiguity by comparing the sequence from two or more sources.

9. The method of claim 6 wherein said provider provides said user with computer executable code for identifying ambiguous sequence within a selected sequence and for resolving the ambiguity of the identified ambiguous sequence.

10. The method of claim 6 wherein said provider also provides said user with the sequence of primers that may be used to amplify said user selected sequence.

11. The method of claim 10 wherein said provider further provides a graphical user interface for said user to order primers with the provided sequence from a third party on the internet.

12. The method of claim 6 wherein said user selected sequence is identified by said user in an association study or a linkage study wherein said sequence is associated with a phenotype.

13. The method of claim 6 wherein said user first identifies said user selected sequence as being associated with a phenotype using a genotyping array that genotypes more than 10,000 different human polymorphisms.

14. The method of claim 6 wherein said sequence computer file is received by the provider over the Internet.

15. The method of claim 6 wherein said design computer file is provided to the user over the Internet.

16. A method for providing resequencing arrays to a user comprising:
using a computer to design one or more resequencing arrays for one or more regions of a genome of interest;
providing users with a list of available designs for resequencing arrays;
receiving an order from a user for a specific design from said list of available designs wherein said specific design is for an array to resequence a selected sequence;
outputting an instruction computer file for said specific design to a nucleic acid synthesizer;
synthesizing an array using said instruction computer file; and
providing the array or arrays to said user.

17. The method of claim 16 further comprising providing primer sequences to the user, wherein said primer sequences may be used to amplify at least one region of said selected sequence.

18. The method of claim 16 wherein said genome of interest is the human genome.

19. The method of claim 16 wherein said selected sequence comprises at least one complete chromosome.

20. The method of claim 16 wherein said list of available designs is provided to said user over the Internet.
